(19) United States
(12) Patent Application Publication
(10) Pub. No.: US 2012/0016327 A1
(43) Pub. Date: Jan. 19, 2012

(54) Method OF Designing Graphics For Absorbent Articles

(76) Inventors: Beth Goldman Mason, Cincinnati, OH (US); Elizabeth Rachelle Mcelroy, Cincinnati, OH (US); Frances J. Thompson, Cincinnati, OH (US)

(21) Appl. No.: 12/835,188

(22) Filed: Jul. 13, 2010

(51) Int. Cl. A61F 13/49; G06F 17/50

(52) U.S. Cl. 604/385.01; 427/2.3; 703/1

(57) ABSTRACT

A method of designing graphics for absorbent articles comprising the steps of: selecting one or more basic technical flats comprising a plurality of printing zones; selecting a color palette; selecting one or more patterns and/or solid prints; and modeling the one or more selected patterns and/or solid prints, using the one or more selected colors, in the printing zones of the basic technical flat that they are selected for, to form an enhanced technical flat that comprises the graphic.

### FIELD OF THE INVENTION

In one aspect, the invention relates generally to methods of designing graphics for absorbent articles and for selling those articles. The method of designing builds upon re-usable design templates with color palettes, patterns, and designs to create a graphic that can be applied to an absorbent article. The method of selling provides co-packaged sets of absorbent articles bearing similar and/or related graphics.

### BACKGROUND OF THE INVENTION

Absorbent articles are used commonly. In many cases, e.g. diapers, the purchaser of the absorbent article is not the end user of the article. Many factors influence the purchaser's decision to purchase particular absorbent articles, non-limiting examples of which include: price and product performance, e.g. absorbency, ease of fastening/refastening, tactile feel against the skin, etc. The aesthetics of an absorbent article have become an increasingly important factor in driving purchase decision-making. Many purchasers prefer to have colors, patterns, and/or other designs employed by the absorbent article. This creates a need to provide desirable graphics without having an unacceptably negative impact on other decision factors, such as price and absorbency.

Conventional methods of designing graphics for absorbent articles may constitute contemplating then adding a border, or simple repeating patterns, e.g. circles or hatch marks, or the method may go so far as to have a graphic designer create a particular design for use with an absorbent article. However, many of these aforementioned methods suffer from one or more disadvantages. For example, the patterns must oftentimes be very simple, e.g. borders, spots, or solid colors, or the patterns take a long time and much effort to get from artistic conception to technical achievement of printing on the absorbent article, and even then, only to yield one or a few designs. Further, typically for a new design, the process must begin from scratch. There is a need to provide more complex and more artistic graphics using less complex and time consuming methods.

Conventional methods of selling absorbent articles typically provide a plurality of absorbent articles in a unit, e.g. polybag or cardboard box, where all the absorbent articles in that unit have identical graphics, if any at all. This method suffers from one or more disadvantages. As noted above, purchasers increasingly prefer absorbent articles with graphics, and it is believed that some purchasers would prefer absorbent articles with a variety of graphics. There is a need to provide absorbent articles with a variety of graphics, preferably similar and/or related graphics, in a single unit or at least units available for purchase together.

### SUMMARY OF THE INVENTION

In one aspect, the invention relates to a method of designing graphics for absorbent articles comprising the steps of: selecting one or more basic technical flats comprising a plurality of printing zones; selecting a color palette; selecting one or more patterns and/or solid prints; and modeling the one or more selected patterns and/or solid prints, using the one or more selected colors, in the printing zones of the basic technical flat that they are selected for, to form an enhanced technical flat that comprises the graphic.

It is an object of this invention to provide a method of designing graphics that allows for complex and numerous graphics. It is another object of this invention to provide a method of designing graphics that allows for more artistic graphics. It is another object of this invention to streamline the process from artistic conception of graphics to technical achievement of printed graphics. These and other objects, features, and advantages of the invention will become more apparent upon reading the following specification in conjunction with the accompanying drawing figures.

### BRIEF DESCRIPTION OF DRAWINGS

FIG. 1 is a flowchart of a method of designing graphics for absorbent articles according to the invention.

FIG. 2 is a perspective view of a basic technical flat employed in an embodiment of the invention.

FIG. 3 is a perspective view of an enhanced technical flat employed in an embodiment of the invention.

FIG. 4 is a perspective view of a croquis employed in an embodiment of the invention.

FIGS. 5a and 5b are perspective views of a pair of croquis employed in an embodiment of the invention.

FIGS. 6a and 6b are perspective views of two graphics employed in an embodiment of the invention.

FIGS. 7a, 7b, and 7c are perspective views of three graphics employed in an embodiment of the invention.

### DETAILED DESCRIPTION OF THE INVENTION

In one aspect, the invention is intended primarily for use with absorbent articles. As used herein, the term “absorbent article” refers to devices which absorb and contain body exudates, and, more specifically, refers to devices which are placed against or in proximity to the body of the wearer to absorb and contain the various exudates discharged from the body. As used herein, the term “diaper” refers to an absorbent article generally worn by infants and incontinent persons that is worn about the lower torso of the wearer. It should be understood, however, that the invention is also applicable to other absorbent articles such as incontinence briefs, incontinence undergarments, incontinence pads, diaper holders and liners, feminine hygiene garments, feminine hygiene pads, feminine hygiene pantiliners, tampons, and the like. As used herein, the term “disposable” refers to absorbent articles that generally are not intended to be laundered or otherwise restored or reused as absorbent articles, i.e. they are intended to be discarded after a single use, and preferably, to be recycled, composted, or otherwise disposed of in an environmentally compatible manner. In an embodiment, the absorbent articles for which the invention is primarily intended for use, are disposable absorbent articles.

In an embodiment of the invention, the method described herein is used to create a graphic for an absorbent article. In another embodiment, the method is used to create several similar and/or related graphics for a set of absorbent articles. In yet another embodiment, the absorbent articles forming such set are offered for sale together as a set.

Absorbent articles may typically comprise a topsheet having a bodyfacing surface and a garment facing surface, a backsheet having a bodyfacing surface and a garment facing surface, and an absorbent core disposed between the garment facing surface of the topsheet and the bodyfacing surface of the backsheet. In an embodiment, the graphics created by the invention are disposed upon the bodyfacing surface of the topsheet. In another embodiment, the graphics are disposed upon the garment facing surface of the topsheet. In another embodiment, the graphics are disposed upon the garment facing surface of the backsheet. In yet another embodiment, the graphics are disposed upon both the bodyfacing surface of the topsheet and the garment facing surface of the backsheet. Other absorbent articles, e.g. catamenial tampons, may typically comprise a compressed absorbent core disposed within an overwrap substantially covering the exterior surface of the compressed absorbent core, the overwrap having a core facing surface and a bodyfacing surface. In an embodiment, the graphics created by the invention are disposed upon the core facing surface of the overwrap. In another embodiment, the graphics are disposed upon the bodyfacing surface of the overwrap. In yet another embodiment, the graphics are disposed upon both the core facing surface of the overwrap and the bodyfacing surface of the overwrap. In an embodiment, the overwrap comprises a non-woven material.

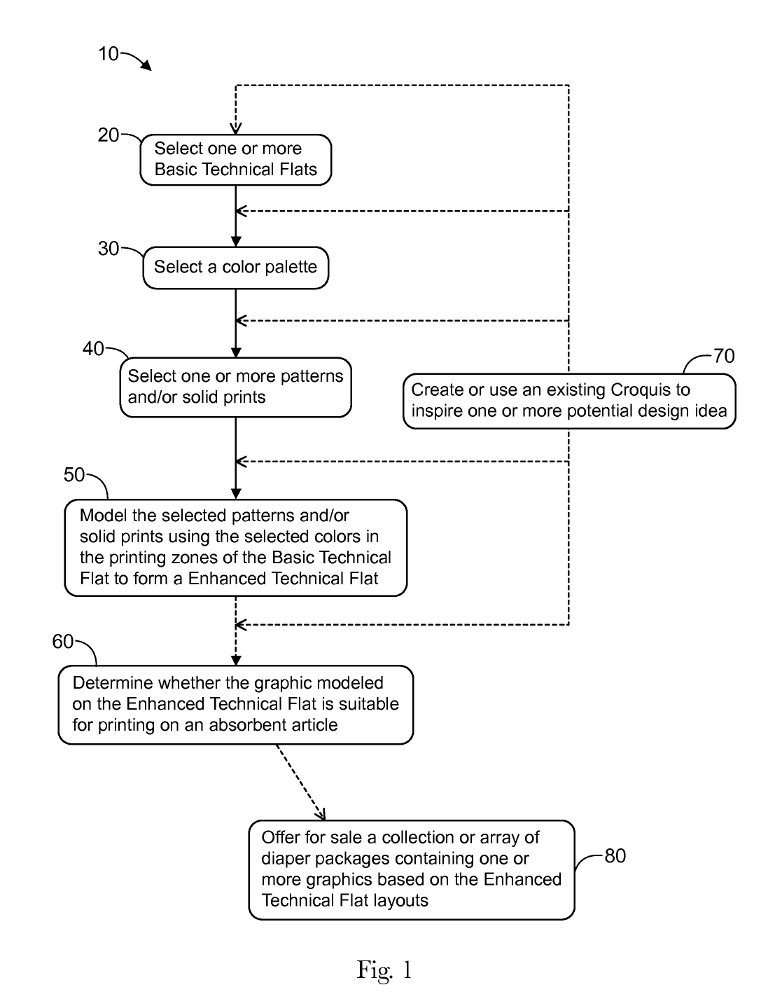

As shown in FIG. 1, a method of designing graphics for absorbent articles 10 is provided. According to method 10, in step 20, one or more basic technical flats 90 is selected, in step 30, a color palette is selected, in step 40, one or more patterns and/or solid prints is selected, in step 50, the selected patterns and/or solid prints, using the selected colors, are modeled in the printing zones 94 of the basic technical flat 90 to form an enhanced technical flat 100. At this point, a graphic for an absorbent article has been designed. These steps and additional optional steps are discussed in more detail herein. It is to be noted that, as depicted in FIG. 1, an embodiment provides that step 30 occurs prior to step 40. However, it will be appreciated by the skilled person that in another embodiment, step 40 occurs prior to step 30, and in yet another embodiment, steps 30 and 40 are performed concurrently.

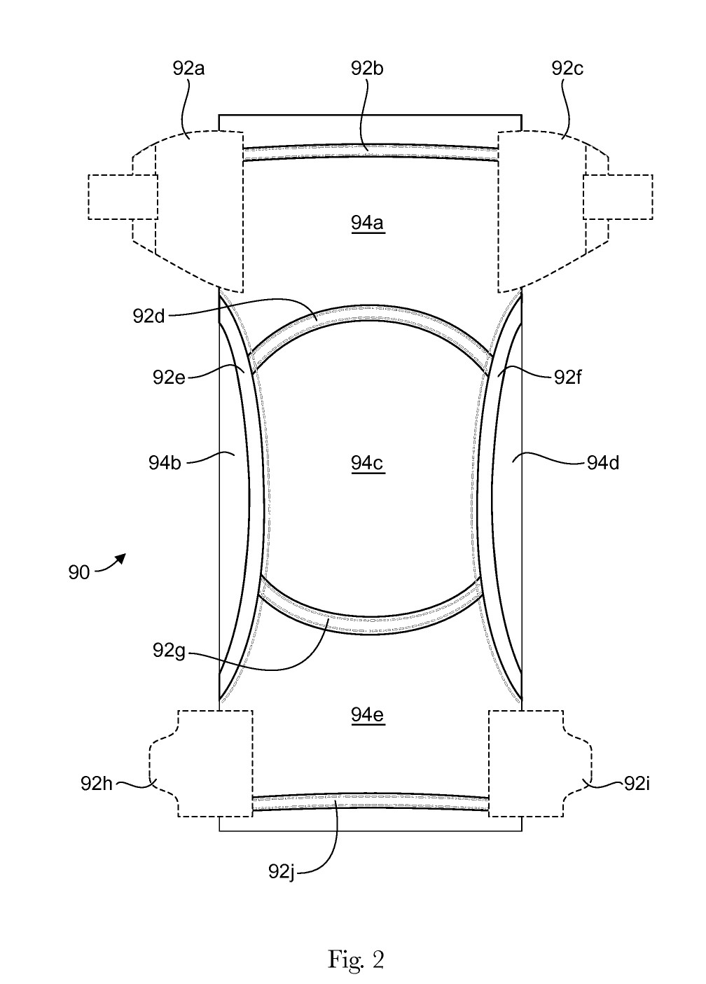

In step 20, one or more basic technical flats 90 is selected. A basic technical flat 90 is a drawing showing the general outline and visible physical features of an absorbent article, a non-limiting example of which is shown in FIG. 2. A basic technical flat 90 may be hand drawn, but is typically mechanically or digitally drawn, thus making it easier to store, transfer to others, and reproduce in mass. A basic technical flat 90 will typically show design elements 92 of the absorbent article. Design elements 92 may be physical features of the absorbent article, such as the overall outline, location of tabs, fasteners, borders, junctions of materials, stitching, and like elements. A basic technical flat 90 may also show some design elements 92 that are either actual physical features of the absorbent article or that are not actual physical features but are to be printed on the absorbent article to make it appear that they are actual elements of the absorbent article, non-limiting examples of features that may fall within either or both of these groups include: printing borders, seams, pockets, zippers, zipper flaps, topstitching, embossment, quilting, buttons, bows, ribbons, straps, snaps, belt loops, suspenders, sales tags, etc. Basic technical flat 90 comprises defined printing zones 94 for which colors and patterns and/or solid prints will be selected for in later steps. Such printing zones 94 may optionally be shaded, hatched, or otherwise detailed to impart an appearance of being 3-dimensional. Typically, a basic technical flat 90 will comprise a plurality of printing zones 94, depending on how many permutations are desired to be available for selection in the final graphic. The printing zones 94 define areas where graphics will be printed on the absorbent articles. Preferably, the basic technical flat 90 comprises from 2 to 8, more preferably from 4 to 6, printing zones. The basic technical flat shown in FIG. 2 has 10 design elements 92a to 92j, and 5 printing zones 94a to 94e.

In step 20, a single basic technical flat 90 may be selected, or to increase the number and variety of graphics being created at once, several basic technical flats 90 may be selected. For example, the selection of multiple basic technical flats 90 may enable one designer to progress several artistic creations concurrently, thereby avoiding the stymie of creative thought that may be associated with following a one-process-at-a-time approach. Similarly, the selection of multiple basic technical flats 90 may enable several designers to progress several artistic creations concurrently and yield a variety of graphics for comparison and/or advancement to finality. Where multiple basic technical flats 90 are available, they will typically exhibit different features of the same absorbent article for which graphics are being designed, or they will exhibit features of different absorbent articles for which graphics are being designed. Accordingly, it is contemplated that similar and/or related graphics could be designed using method 10 for a variety of different absorbent articles in a product line, e.g. diapers and pants, or feminine pads and pantiliners.

In step 30, a color palette is selected. Preferably, the colors are complementary to each other and to the patterns and/or solid prints selected in step 40. By complementary, it is meant that a designer believes they work together to achieve a common artistic theme or expression, noting that such theme may be one of intentional contrast. It is not intended that they must be classically accepted by the general population as “going together”. For example, a designer may select a color palette in which the colors appear to clash to the ordinary observer, but upon further investigation, reveal a psychedelic color scheme.

In step 40, one or more patterns and/or solid prints is selected. Preferably, the patterns and/or solid prints are complementary to each other and to the colors selected in step 30. As with the colors, complementary means that the designer believes that they work together to achieve a common artistic theme or expression, noting that such theme may be one of intentional contrast. In an embodiment, the patterns and/or solid prints are obtained from a print house, a print designer, or a fabric pattern designer.

For each of steps 30 and 40, the colors and the pattern and/or solid print, respectively, may be independently selected from themed or otherwise like groups of colors and patterns and/or solid prints. In an embodiment, they may be selected from groups being categorized as being gender neutral, boy appropriate, or girl appropriate. In another embodiment, they may be selected from groups categorized as being sporty, outdoors, sophisticated, professional, casual, cute, sassy, feminine (e.g. quilted, paisley, curly cues, polka dots), fresh, seasonal (e.g. spring, summer, fall, winter), patriotic, weather/climatic (e g sunny, rainy, snowy), ethnic, soft tones, earth tones, pastels, rock 'n roll, western (e.g. cowboy/cowgirl), animal, plant, food, or industrial.

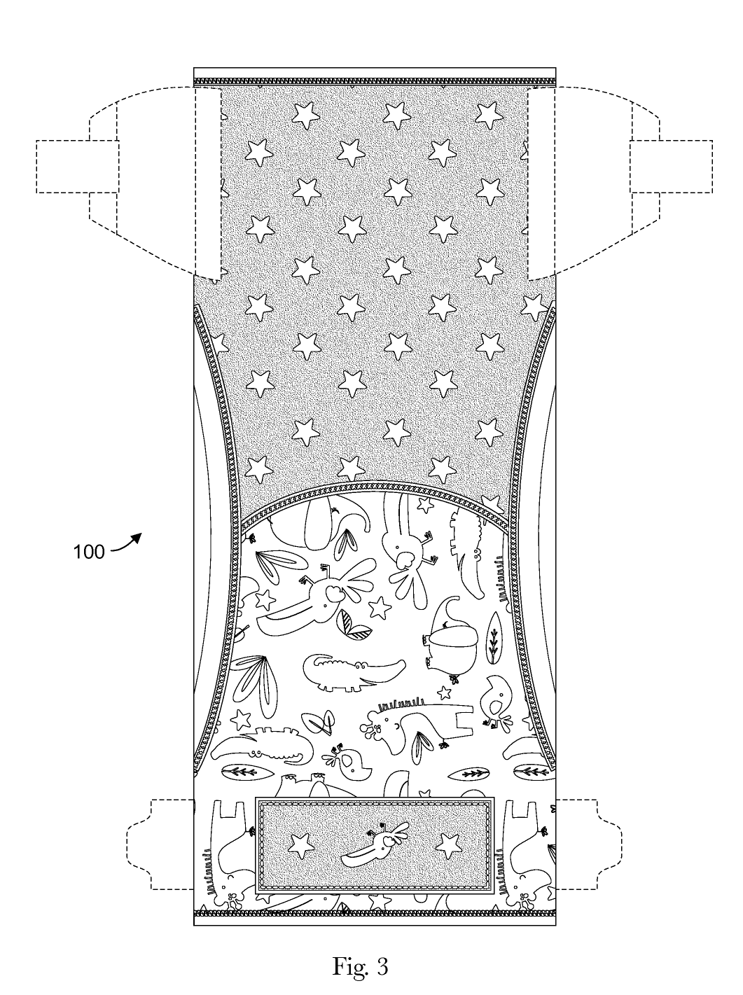

In step 50, the selected patterns and/or solid prints, using the selected colors, are modeled in the printing zones 94 of the basic technical flat 90 to form an enhanced technical flat 100, as shown in FIG. 3, where color is depicted by shading. An enhanced technical flat 100 is a basic technical flat 90 upon which the designer's selections in steps 30 and 40 are matched up with the printing zone that the designer selected them for, whereby the full graphic is created. Modeling may be achieved by such simple means as placing hard copy colored patterns and/or solid prints onto a hard copy basic technical flat 90, or may be achieved by more technical means, including printing on paper or another substrate including materials used in absorbent articles, and/or computer modeling. Modeling may also be achieved by creating a physical prototype of the absorbent article exhibiting all or a portion of the graphic. Any means sufficient for appreciating the final graphic may be used.

Optionally, step 60 may follow step 50. In step 60, a determination is made whether the graphic modeled on the enhanced technical flat 100 is suitable for printing on an absorbent article. It will be appreciated that there are various and many technical hurdles to be overcome when transferring a graphic from the design stage to actually disposing it upon, e.g. printing it onto, an absorbent article, a non-limiting example of which is the difficulty in registering the design to the portion of the material that it is being printed on. It will also be appreciated that due to the wide range of expression and interpretation of such expression in the fashion realm, a graphic that appears suitable to one person may not appear suitable to another. As such, there are many reasons why a graphic may need to be revised before becoming final. In the event that it is determined in step 60 that the graphic requires revision, then the graphic may be returned to any of steps one of steps 20, 30, 40, 50, or 60, of method 10.

Advantageously, due to the unexpected “assembly line” nature of method 10, unexpected at least because the designing process in the fashion realm is not intuitively a good candidate for processes typically applied to more mechanical or technical actions, the graphic may be revised to overcome the objectionable feature relatively quickly and without significant re-work. For example, if the graphic created in step 50 is of acceptable pattern but objectionable color, the designer may return to step 30 and consider selecting other color palettes, and then proceed with the remaining steps of method 10 again until a modified graphic is created in step 50. Then, step 60 may be reapplied and a new determination made of whether this modified graphic is acceptable, and so on. Yet another advantage of method 10 is that the work product created at each step may be saved for future reference or use, again minimizing re-work, and hence, saving time and cost.

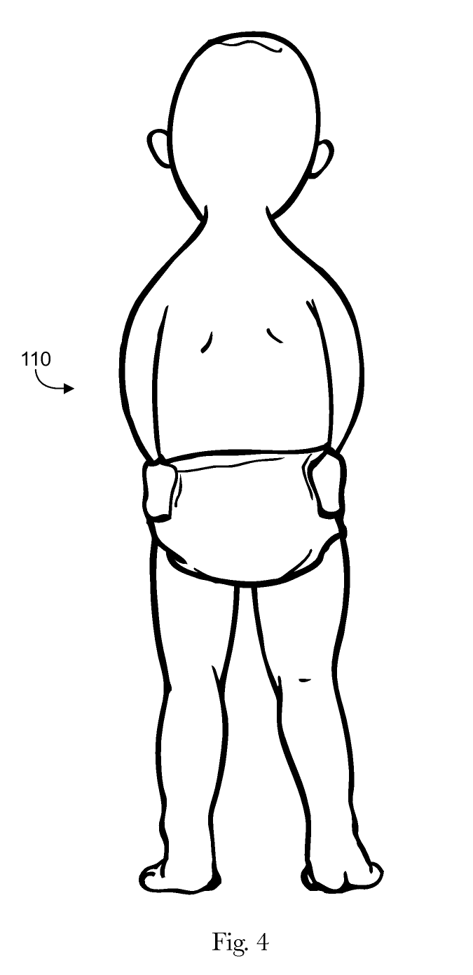

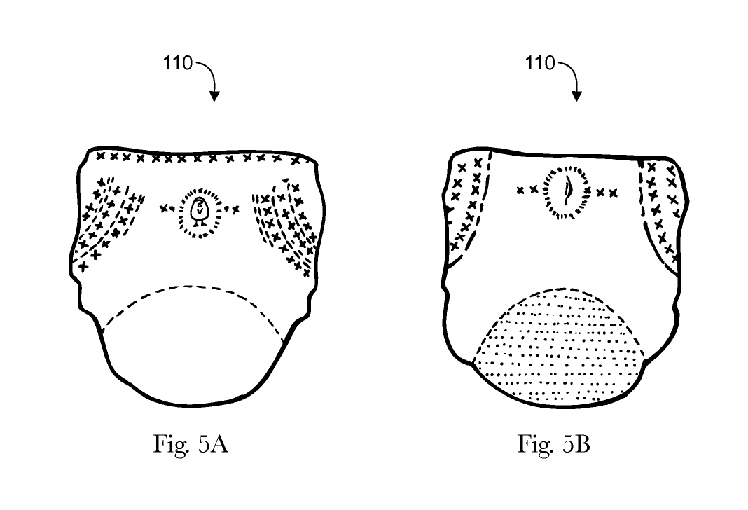

Optionally, step 70 may precede any one of steps 20, 30, 40, 50, or 60, of method 10. In step 70, a croquis is created or an existing croquis is used, to inspire one or more potential design ideas. A croquis 110 is a sketch showing very general outline and few basic physical features of an absorbent article, non-limiting examples of which are shown in FIGS. 4, 5a, and 5b. A croquis 110 is typically hand drawn, but may be mechanically or digitally drawn, thus making it easier to store, transfer to others, and reproduce in mass. A croquis 110 will typically show such features of the absorbent article as the overall outline, the location of only a few features, e.g. fasteners, borders, and like elements. A croquis 110 may show a simple version of the wearer of the absorbent article, as is shown in FIG. 4. Alternately, a croquis 110 may omit the wearer and focus more on the basic views, e.g. front and back as worn, of the absorbent article without regard to many technical features, as is shown in FIGS. 5a and 5b. A croquis 110 may be in black and white, or in color, and either way, may optionally be shaded, hatched, or otherwise detailed to impart an appearance of being 3-dimensional.

The skilled person will create or select a created croquis 110 with sufficient detail about the features of the absorbent article corresponding to the step of method 10 for which the croquis 110 is being used to inspire one or more potential design ideas. For example, a croquis 110 used prior to step 20 may look more like the croquis 110 shown in FIG. 4, which provides very little detail about the features of the absorbent article, e.g. the general shape of the absorbent article when seen from the back of the wearer, the location of two fasteners, and some shading to impart 3-dimensional appearance. However, a croquis 110 used prior to either or both of steps 30 and 40 may look more like the croquis 110 shown in FIGS. 5a and 5b, which still provide little detail

about the features of the absorbent article, e.g. a 2-dimensional view of the general shape of the absorbent article when seen from both the front and back of the wearer, without the wearer present, and the location of two fasteners, but do provide more detail about the possible colors and patterns that may be employed in the ultimately designed graphic.

The skilled person will appreciate that absorbent articles bearing similar and/or related graphics may be advantageously designed by using method 10. A method of selling such articles is disclosed in commonly assigned, co-pending U.S. Ser. No. XX/XXX,XXX, entitled “Method of Selling Absorbent Articles Bearing Similar and/or Related Graphics”, filed on 13 Jul., 2010, and having Attorney Docket No. 11797. As used herein, “similar and/or related” refers to a degree of sameness of at least some of the elements of the graphics embodied two or more absorbent articles. It is not intended to mean that two or more graphics being compared are identical except for scale, e.g. due to the sizes of the various absorbent articles being different. Elements to consider in determining similarity and/or relatedness include but are not limited to: color palettes (such as described in step 30 of method 10), patterns and/or solid prints (such as described in step 40 of method 10), shapes, themes, voids, position, orientation, and the like; preferably color palettes and patterns and/or solid prints are considered.

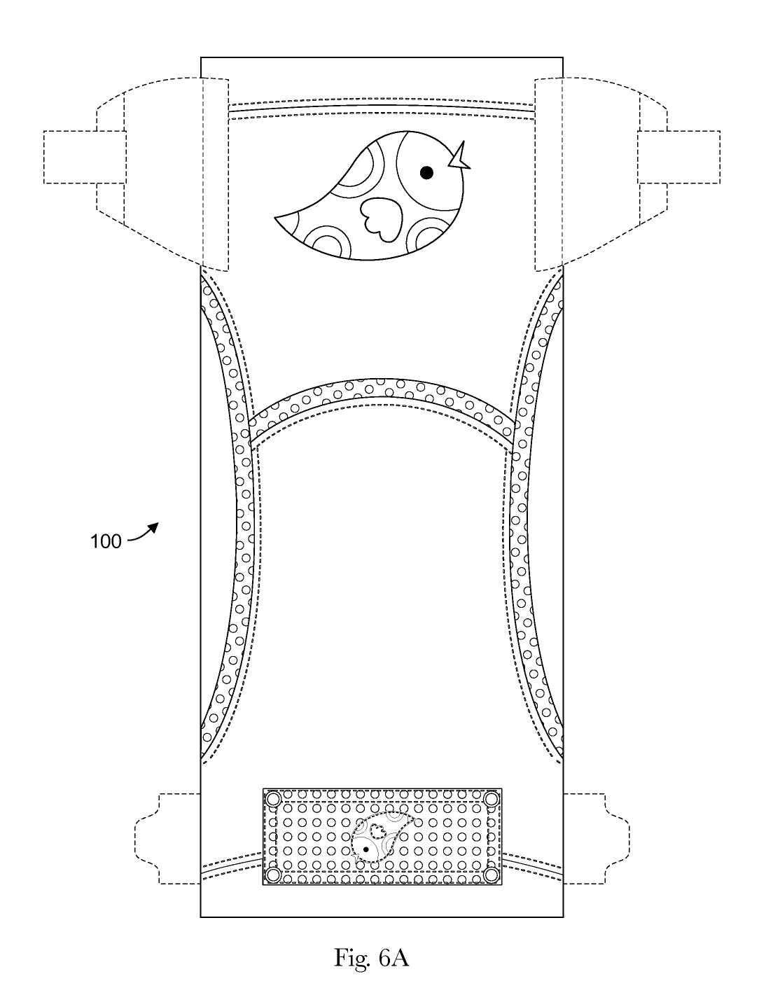

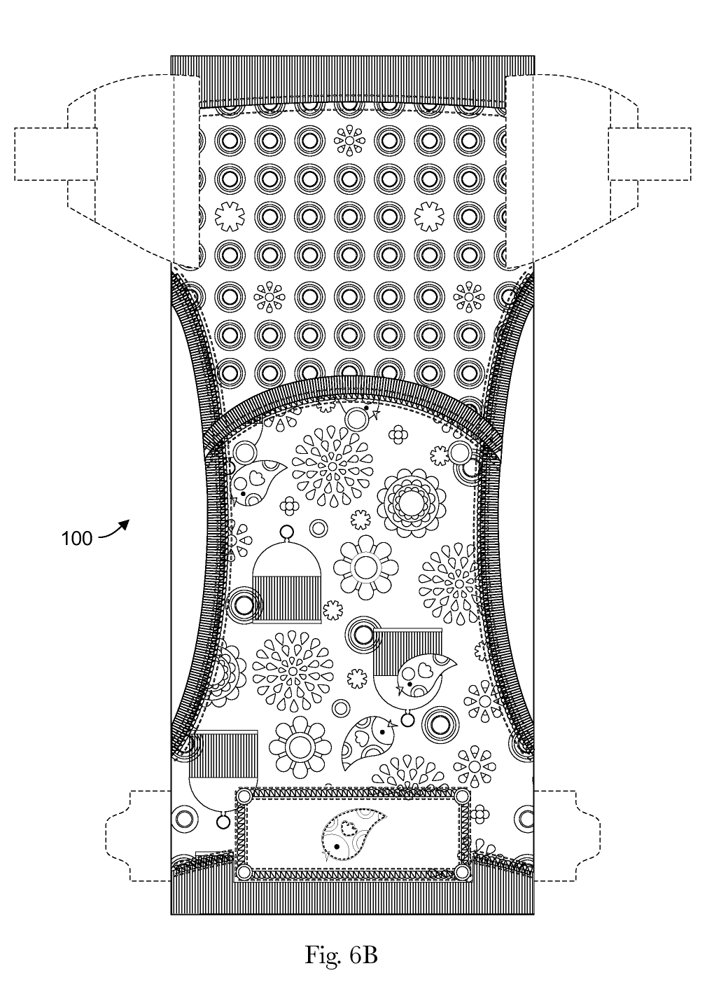

As shown in FIGS. 6a and 6b, graphics that are similar and/or related may share a common color palette, common elements, e.g. birds, common positioning of areas that are printed vs. non-printed, and common placement of actual or apparent stitching. In an embodiment, as shown in FIGS. 6a and 6b, the array of absorbent articles may bear similar and/or related graphics where one absorbent article (e.g. one bearing the graphic shown in FIG. 6a) may exhibit a simple and plain look about it, e.g. having a significant amount of unprinted space and basic graphics, whereas another absorbent article (e.g. one bearing the graphic shown in FIG. 6b) may exhibit substantially more complex graphics that include additional patterns not seen in the other absorbent article, but still keeping within, e.g. the same theme. Such an array may be characterized as a “collection” of absorbent articles.

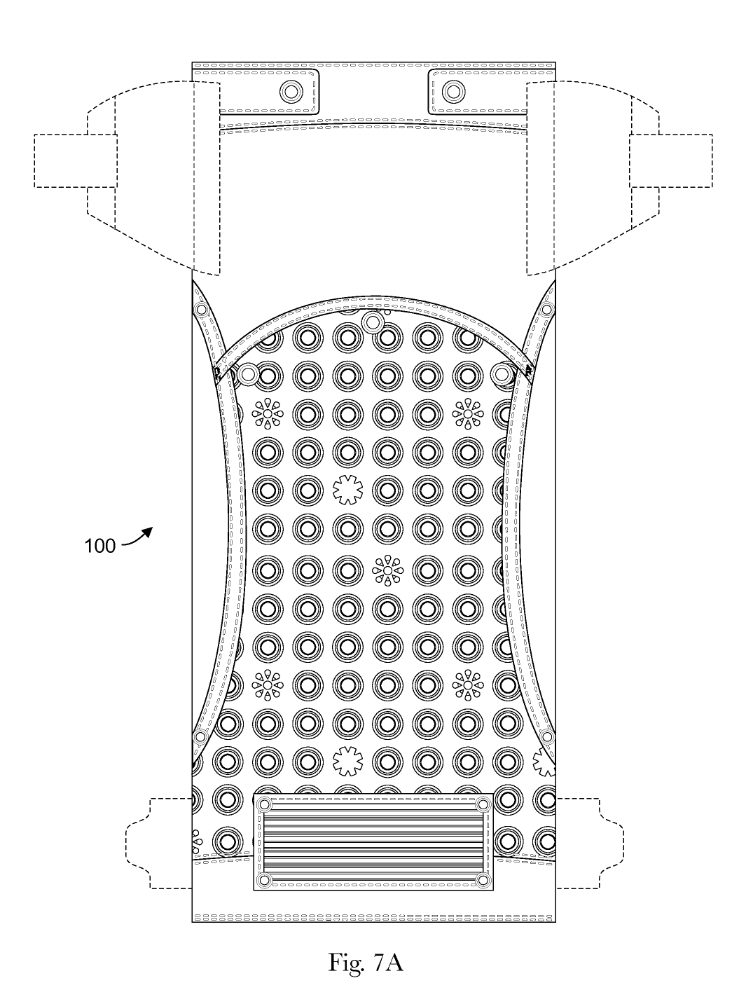

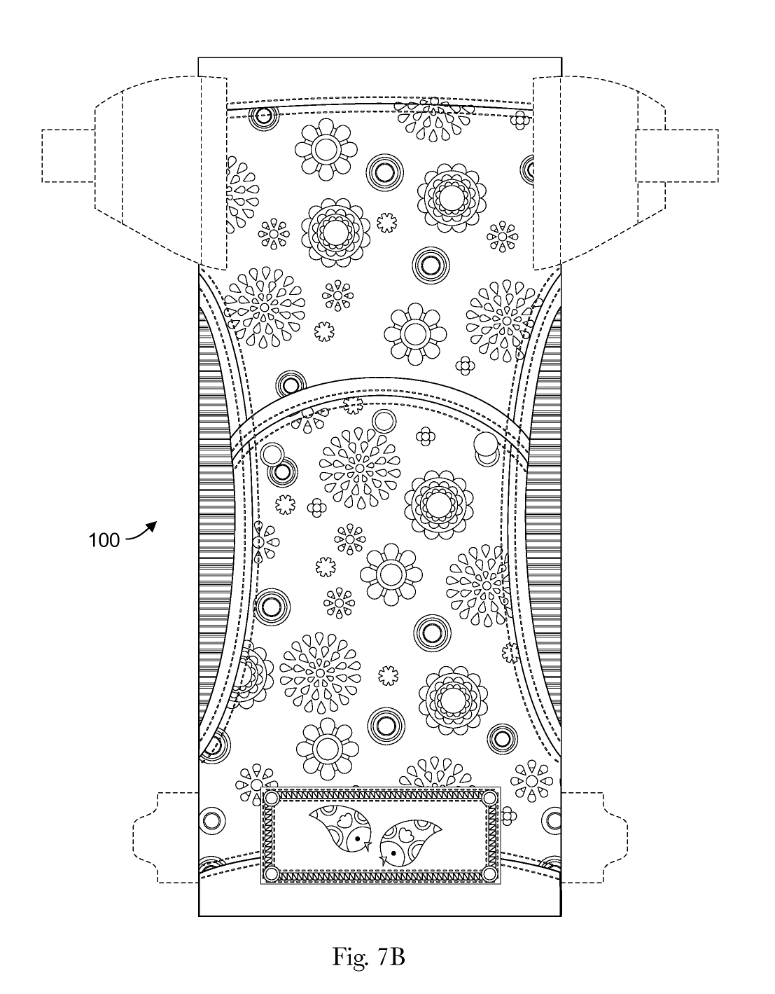

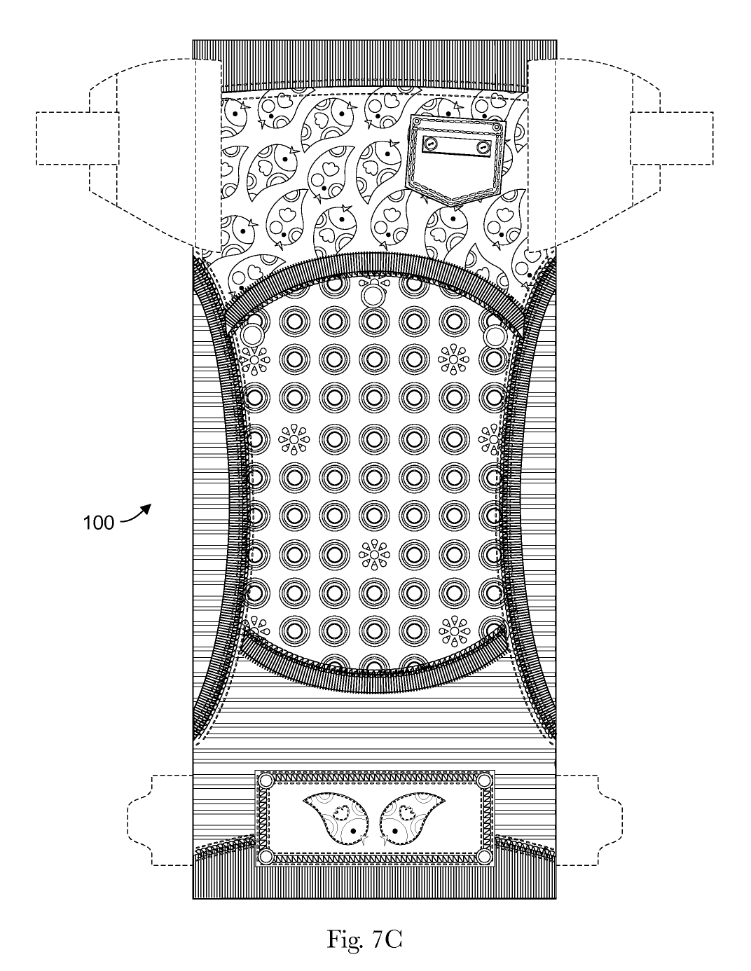

As shown in FIGS. 7a, 7b, and 7c, graphics that are similar and/or related may share a common color palette, common use of concentric circles, common lined patterns, common elements, e.g. birds, and common positioning of areas that are printed vs. non-printed. In another embodiment, as shown in FIGS. 7a, 7b, and 7c, the array of absorbent articles may bear similar and/or related graphics where one absorbent article (e.g. one bearing the graphic shown in FIG. 7a) may exhibit a simple look about it, e.g. having two main patterns with some bordering, whereas another absorbent article (e.g. one bearing the graphic shown in FIG. 7b) may exhibit somewhat more complex graphics that include additional patterns not seen in the other absorbent article, but still keeping within, e.g. the same theme, and yet another absorbent article (e.g. one bearing the graphic shown in FIG. 7c) may exhibit yet more complex graphics that combine various elements from the other two absorbent articles and added a further feature, e.g. the pocket, but still keeping within, e.g. the same theme. It is to be noted that the color palette is common across all three absorbent articles. Such an array may also be characterized as “collection” of absorbent articles. Collections of absorbent articles comprise a plurality of design graphics, such as those created using method 10.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of designing graphics for absorbent articles comprising the steps of:
a. selecting one or more basic technical flats comprising a plurality of printing zones;
b. selecting a color palette;
c. selecting one or more patterns and/or solid prints; and
d. modeling the one or more selected patterns and/or solid prints, using the one or more selected colors, in the printing zones of the basic technical flat that they are selected for, to form an enhanced technical flat that comprises the graphic.

2. The method of claim 1, wherein the order of performance of steps b and c are selected from the group consisting of: (i) b, then c; (ii) c, then b; and (iii) b and c are performed concurrently.

3. The method of claim 1, wherein the one or more basic technical flat shows at least one actual or apparent feature of the absorbent article selected from the group consisting of: overall outline, location of tabs, fasteners, borders, junctions of materials, stitching, printing borders, seams, pockets, zippers, zipper flaps, topstitching, embossment, quilting, buttons, bows, ribbons, straps, snaps, belt loops, suspenders, and sales tags.

4. The method of claim 1, wherein at least one of the printing zones is detailed to detailed to impart an appearance of being 3-dimensional.

5. The method of claim 1, wherein the one or more basic technical flat comprises from about 2 to about 8 printing zones.

6. The method of claim 1, wherein more than one basic technical flat is selected.

7. The method of claim 1, wherein one or more patterns are selected.

8. The method of claim 1, wherein one or more solid prints are selected.

9. The method of claim 1, wherein the one or more the patterns and/or solid prints are obtained from a print house, a print designer, or a fabric pattern designer.

10. The method of claim 1, wherein at least one of the one or more colors and one or more patterns and/or solid prints are independently selected from groups categorized as being: gender neutral, boy appropriate, girl appropriate, sporty, outdoors, sophisticated, professional, casual, cute, sassy, feminine, quilted, paisley, curly cues, polka dots, fresh, seasonal, spring, summer, fall, winter, patriotic, weather, climatic, sunny, rainy, snowy, ethnic, soft tones, earth tones, pastels, rock 'n roll, western, cowboy, cowgirl, animal, plant, food, industrial, and combinations thereof.

11. The method of claim 1, wherein a similar and/or related graphics are designed for a variety of different absorbent articles in the same product line.

12. The method of claim 1, wherein the absorbent articles are diapers.

13. The method of claim 1, wherein the absorbent articles are feminine pads, pantiliners, or tampons.

14. The method of claim 1, wherein the absorbent articles are incontinence pads.

15. The method of claim 1, further comprising the step of determining whether the graphic modeled on the enhanced technical flat is suitable for printing on an absorbent article.

16. The method of claim 15, wherein it is determined that the graphic modeled on the enhanced technical flat is not suitable for printing on an absorbent article and the graphic is returned to any step of a method of designing graphics for absorbent articles comprising the steps of :
a. selecting one or more basic technical flats comprising a plurality of printing zones;
b. selecting a color palette;
c. selecting one or more patterns and/or solid prints;
d. modeling the one or more selected patterns and/or solid prints, using the one or more selected colors, in the printing zones of the basic technical flat that they are selected for, to form an enhanced technical flat that comprises the graphic.

17. The method of claim 1, further comprising the step of creating and using a croquis or using an existing croquis to inspire one or more potential design ideas.

18. The method of claim 17, wherein the croquis comprises a depiction of a wearer of the absorbent article.

19. The method of claim 17, wherein the croquis does not comprise a depiction of a wearer of the absorbent article.

20. The method of claim 17, wherein at least a portion of the croquis is detailed to impart an appearance of being 3-dimensional.

21. The method of claim 15, further comprising the step of creating and using a croquis or using an existing croquis to inspire one or more potential design ideas.

22. An absorbent article comprising a topsheet having a bodyfacing surface and a garment facing surface, a backsheet having a bodyfacing surface and a garment facing surface, and an absorbent core disposed between the garment facing surface of the topsheet and the bodyfacing surface of the backsheet, wherein a graphic designed by the method of claim 1 is disposed upon a surface of the absorbent article selected from the group consisting of: the bodyfacing surface of the topsheet, the garment facing surface of the topsheet, the garment facing surface of the backsheet, and both the bodyfacing surface of the topsheet and the garment facing surface of the backsheet.

23. An absorbent article comprising a compressed absorbent core disposed within an overwrap substantially covering the exterior surface of the compressed absorbent core, the overwrap having a corefacing surface and a bodyfacing surface, wherein a graphic designed by the method of claim 1 is disposed upon a surface of the absorbent article selected from the group consisting of: the corefacing surface of the overwrap, the bodyfacing surface of the overwrap, and both the corefacing surface of the overwrap and the bodyfacing surface of the overwrap.
